(19) United States
(12) Patent Application Publication
(10) Pub. No.: US 2026/0177550 A1
(43) Pub. Date: Jun. 25, 2026

(54) CLINICAL APPLICATION OF SBCMA IN CEREBROSPINAL FLUID IN DIAGNOSIS AND MONITORING OF CENTRAL NERVOUS SYSTEM AUTOIMMUNE DISEASES

(71) Applicant: TONGJI HOSPITAL,TONGJI MEDICAL COLLEGE,HUAZHONG UNIVERSITY OF SCIENCE AND TECHNOLOGY, Wuhan (CN)

(72) Inventors: Chuan QIN, Wuhan (CN); Daishi TIAN, Wuhan (CN); Wei WANG, Wuhan (CN); Xiaowei PANG, Wuhan (CN); Yang YU, Wuhan (CN)

(73) Assignee: TONGJI HOSPITAL,TONGJI MEDICAL COLLEGE,HUAZHONG UNIVERSITY OF SCIENCE AND TECHNOLOGY, Wuhan (CN)

(21) Appl. No.: 19/377,048

(22) Filed: Nov. 3, 2025

Related U.S. Application Data
(63) Continuation of application No. PCT/CN2025/073879, filed on Jan. 22, 2025.

(30) Foreign Application Priority Data
Dec. 19, 2024 (CN) 202411885246.1

(51) Int. Cl. G01N 33/564; G01N 33/68

(52) U.S. Cl. CPC G01N 33/564; G01N 33/6863; G01N 2333/70578; G01N 2800/24

(57) ABSTRACT

Disclosed is an application of sBCMA as a biomarker in cerebrospinal fluid in diagnosis and monitoring of antibody-mediated central nervous system autoimmune diseases. Taking NMOSD as an example, the level of sBCMA in cerebrospinal fluid of NMOSD patients is significantly higher than that of control subjects matched for age and gender, and there is a positive correlation between the level of sBCMA in cerebrospinal fluid and a nerve injury-related index NFL, a blood-cerebrospinal fluid barrier breakdown-related index QAlb, or a neuroinflammation-related index sTREM2 in cerebrospinal fluid of NMOSD patients. This indicates that the level of sBCMA in cerebrospinal fluid can be used as a biomarker for the diagnosis of antibody-mediated central nervous system autoimmune diseases such as NMOSD and the evaluation of the severity of nerve injuries, the degree of damage to blood-cerebrospinal fluid barrier, and the level of neuroinflammation in patients.

### CROSS-REFERENCE TO RELATED APPLICATIONS

The application claims priority to Chinese patent application No. 2024118852461, filed on Dec. 19, 2024, the entire contents of which are incorporated herein by reference.

### TECHNICAL FIELD

This disclosure belongs to the technical field of disease detection, and specifically relates to an application of sBCMA as a biomarker in cerebrospinal fluid in diagnosis and monitoring of antibody-mediated central nervous system autoimmune diseases.

### BACKGROUND

Autoimmune diseases are a group of diseases characterized by immune dysfunction and abnormal immune response to normal body composition from the body. Humans have discovered more than 80 autoimmune diseases, including systemic diseases, such as systemic lupus erythematosus, and also including tissue or organ-specific diseases, such as autoimmune thyroiditis. Autoimmune diseases can widely affect all systems of the body, including skin, joints, kidneys, and the central nervous system.

Central nervous system (CNS) autoimmune diseases are autoimmune diseases, with main pathogenesis of autoimmune cells, autoantibodies, and other immune molecules directly or indirectly attacking the nervous system (including neurons, glial cells, and myelin sheath). There are many kinds of CNS autoimmune diseases, including neuromyelitis optica spectrum disorders (NMOSD), myelin oligodendrocyte glycoprotein-IgG associated disorders (MOGAD), multiple sclerosis (MS), acute disseminated encephalomyelitis (ADEM), autoimmune encephalitis (AE), CNS vasculitis, etc. The pathogenesis of the CNS autoimmune diseases is complex, and immune inflammatory responses caused by direct or indirect attacks on the nervous system by the autoimmune cells, autoantibodies, and other immune molecules play an important role in the pathogenesis. The autoantibodies play a very important role in the diagnosis and differential diagnosis of CNS inflammatory demyelinating diseases.

B lymphocytes/plasma cells are one of the most important effector cells of autoimmune diseases, which attack normal cells, tissues, and organs through abnormal secretion of autoantibodies, and induce the body to produce systemic immune abnormalities. The proliferation and maturation of B lymphocytes play an important role in the pathogenesis of the CNS autoimmune diseases. The B-cell maturation antigen (BCMA) is a transmembrane glycoprotein in the tumor necrosis factor superfamily, and is mainly expressed on the surface of mature B lymphocytes. It regulates the maturation and differentiation of B cells into plasma cells. The soluble B-cell maturation antigen (sBCMA) is directly detached from the membrane BCMA, and has been proved to be a good biomarker for multiple myeloma and systemic lupus erythematosus. In a prospective study, the correlation between sBCMA of serum and the degree of infiltration of plasma cells of bone marrow in patients with multiple myeloma was evaluated, which proved that the level of sBCMA was a good prognostic index for multiple myeloma. In another retrospective cohort study, the potential and significance of the sBCMA of serum as a biomarker in patients with systemic lupus erythematosus were revealed. Due to the unique structure of CNS, blood-brain barrier, autoimmune cells cannot easily enter the CNS under normal circumstances. Therefore, different from other autoimmune diseases, the immune responses of antibody-mediated CNS autoimmune diseases may be limited, and there is no relevant clinical study showing the clinical application of sBCMA as a biomarker in the diagnosis and monitoring of antibody-mediated CNS autoimmune diseases.

### SUMMARY

This disclosure provides new use of sBCMA as a biomarker in cerebrospinal fluid, that is, an application in preparing a diagnostic kit and disease monitoring kit of antibody-mediated central nervous system autoimmune diseases represented by neuromyelitis optica spectrum disorders (NMOSD).

To achieve the foregoing purpose, this disclosure adopts the following technical solution:

An application of a reagent for detecting a biomarker sBCMA in cerebrospinal fluid in preparation of a kit, includes the following applications:
(1) the level of sBCMA in cerebrospinal fluid is detected for diagnosis of antibody-mediated central nervous system autoimmune diseases;
(2) the level of sBCMA in cerebrospinal fluid is detected for evaluating the severity of nerve injuries of patients with antibody-mediated central nervous system autoimmune diseases;
(3) the level of sBCMA in cerebrospinal fluid is detected for evaluating the degree of damage to blood-cerebrospinal fluid barrier of patients with antibody-mediated central nervous system autoimmune diseases; and
(4) the level of sBCMA in cerebrospinal fluid is detected for evaluating the level of neuroinflammation of patients with antibody-mediated central nervous system autoimmune diseases.

Specifically, the following steps are included: a. determining the level of sBCMA in cerebrospinal fluid of subjects; and b. carrying out significant differential analysis by comparing the level of sBCMA in the cerebrospinal fluid of the subjects with the level of sBCMA of control subjects matched for age and gender. There is a positive correlation between the level of sBCMA in cerebrospinal fluid of patients with antibody-mediated central nervous system autoimmune diseases and the level of NFL, QAlb, or the level of sTREM2 in cerebrospinal fluid of patients with antibody-mediated central nervous system autoimmune diseases. Therefore, compared with the level of sBCMA in the control subjects, the higher the level of sBCMA in the cerebrospinal fluid of the subjects reflects the more serious nerve injuries, the more serious the blood-cerebrospinal fluid barrier dysfunction, and the higher the level of neuroinflammation.

In addition to neuromyelitis optica spectrum disorders, the above antibody-mediated nervous system autoimmune diseases also include myelin oligodendrocyte glycoprotein-IgG associated disorders, anti-N-methyl-D-aspartate receptor encephalitis, anti-leucine-rich glioma-inactivated protein 1 encephalitis, and anti-contactin associated protein-like 2 antibody autoimmune encephalitis.

Compared with the prior art, this disclosure has the following advantages and beneficial effects: first, it is confirmed that the level of sBCMA in cerebrospinal fluid of NMOSD patients is obviously higher than that of control subjects matched for age and gender. Further, according to this disclosure, it is confirmed that there is a positive correlation between the concentration of sBCMA in cerebrospinal fluid of NMOSD patients and related indexes of nerve injuries and blood-cerebrospinal fluid barrier breakdown, which indicates that the level of sBCMA in cerebrospinal fluid of NMOSD patients can be used as a biomarker for auxiliary diagnosis of NMOSD patients, and can reflect the severity of nerve injuries and the degree of pathological damage in NMOSD patients. In addition, according to this disclosure, it is also confirmed that there is a positive correlation between sBCMA and neuroinflammation in cerebrospinal fluid of NMOSD patients. Therefore, sBCMA, as a biomarker, can provide powerful auxiliary means for diagnosis and monitoring of NMOSD and other antibody-mediated central nervous system autoimmune diseases (including myelin oligodendrocyte glycoprotein-IgG associated disorders, anti-N-methyl-D-aspartate receptor encephalitis, anti-leucine-rich glioma-inactivated protein 1 encephalitis, and anti-contactin associated protein-like 2 antibody autoimmune encephalitis), with better sensitivity and specificity.

### BRIEF DESCRIPTION OF THE DRAWINGS

FIG. 1 shows the analysis results of the level of sBCMA in cerebrospinal fluid of subjects of different genders;

FIG. 2 shows analysis results of correlation between the age and the level of sBCMA in cerebrospinal fluid of subjects;

FIG. 3 shows analysis results of the level of sBCMA in cerebrospinal fluid of subjects;

FIG. 4 is a ROC curve of the level of sBCMA in cerebrospinal fluid of subjects;

FIG. 5 shows analysis results (A) of the level of a nerve injury-related index NFL of subjects and analysis results (B) of correlation between the level of sBCMA in cerebrospinal fluid and the NFL;

FIG. 6 shows analysis results (A) of the level of a blood-cerebrospinal fluid barrier breakdown-related index QAlb of subjects and analysis results (B) of correlation between the level of sBCMA in cerebrospinal fluid and the QAlb;

FIG. 7 shows analysis results (A) of the level of a neuroinflammation-related index sTREM2 of subjects and analysis results (B) of correlation between the level of sBCMA in cerebrospinal fluid and the neuroinflammation-related index sTREM2;

FIG. 8 shows analysis results of the level of sBCMA in cerebrospinal fluid of subjects of different genders in a validation cohort;

FIG. 9 shows analysis results of correlation between the age and the level of sBCMA in cerebrospinal fluid of subjects in a validation cohort;

FIG. 10 shows analysis results of the level of sBCMA in cerebrospinal fluid of subjects in a validation cohort;

FIG. 11 is a ROC curve of the level of sBCMA in cerebrospinal fluid of subjects in a validation cohort;

FIG. 12 shows analysis results of the level of a nerve injury-related index NFL in cerebrospinal fluid and correlation between the level of sBCMA in cerebrospinal fluid and the NFL of subjects in a validation cohort; and

FIG. 13 shows analysis results of the level of a neuroinflammation-related index sTREM2 in cerebrospinal fluid and correlation between the level of sBCMA in cerebrospinal fluid and the sTREM2 of subjects in a validation cohort.

### DETAILED DESCRIPTION OF THE EMBODIMENTS

Neuromyelitis optica spectrum disorder (NMOSD) is a rare, progressive, autoimmune-mediated CNS inflammatory demyelinating disease mainly involving optic nerve and spinal cord. NMOSD is characterized by inflammatory demyelination and axonal injury of optic nerve and spinal cord, which can cause blindness, paralysis, and incontinence. Its main pathogenesis is as follows: plasma cells derived from B lymphocytes produce aquaporin-4 (AQP4) antibodies, which bind to AQP4 antigens on astrocytes through a blood-brain barrier, and finally causes astrocyte damage, oligodendrocyte damage, demyelination, and neuron loss through mechanisms such as antibody dependent cytotoxicity and complement dependent cytotoxicity. NMOSD is most commonly found on young adults, mostly women. Severe optic neuritis and longitudinally extensive transverse myelitis are the main clinical features, with high recurrence rate and disability rate. In China, the incidence rate is about 0.41/100,000, and the prevalence rate is about 3.31/100,000. NMOSD is a highly recurrent and disabling disease, and most of the subjects have serious sequelae, which brings a great burden to the health system, families, and groups. Taking NMOSD as an example, this disclosure reveals the important role of sBCMA as a biomarker in cerebrospinal fluid in diagnosis and monitoring of antibody-mediated CNS autoimmune diseases.

### Example 1 sBCMA in Cerebrospinal Fluid as a Biomarker of NMOSD

Subjects for clinical study: the subjects for clinical study in the following example included: 30 (aquaporin-4 antibody positive-AQP4-IgG positive) NMOSD patients, and 30 serum AQP4 antibody-negative control subjects matched for gender and age. Specific details are shown in the following table:

| | Control group (n = 30) | NMOSD (n = 30) |
|---|---|---|
| Female, n (%) | 26(86.7) | 27(90.0) |
| Age (years), median | 48.5 (31.0-63.0) | 52.0 (28.0-72.0) |
| (maximum/minimum) | | |

### (1) Sample Collection and Preparation

Sterile centrifuge tubes were used to collect cerebrospinal fluid samples from the subjects for clinical study. The collected cerebrospinal fluid samples were centrifuged at 500×g for 10 minutes at 4° C., and the supernatant was aliquoted into freezing tubes and stored in an ultra-low temperature refrigerator at −80° C.

(2) Method for Detecting Concentration of sBCMA

Human BCMA/TNFRSF17 ELISA kit (R&D Systems, DY193) was adopted to detect the expression levels of sBCMA in cerebrospinal fluid. Specifically, before the detection, the samples were restored to a temperature range of 18° C.-25° C. The samples were diluted and standards were prepared according to the instructions. Next, 100 μl of standards and samples were added to a well plate and incubated for 2.5 hours at room temperature. Subsequently, the liquid in the well plate was discarded and 1× washing liquid was used to wash the plate for 3 times (300 μl each time, 3-5 minutes each time). Then, 100 μl of antibodies were added and incubated for 1 hours at room temperature. Similarly, the washing liquid was used to wash the plate for 3 times. 100 μl of HRP were then added and incubated for 45 minutes at room temperature, and after that, the plate was washed again. 100 μl of substrate solution was added to each well in turn, and color development was carried out for 30 minutes at room temperature in the dark. After the color development, 50 μl of termination solution (2 mol/L of H₂SO₄) was added to each well to terminate the reaction. Finally, an OD value of 450 nm was read by a microplate reader to determine the level of sBCMA in cerebrospinal fluid.

(3) Analysis of the Level of sBCMA in Cerebrospinal Fluid

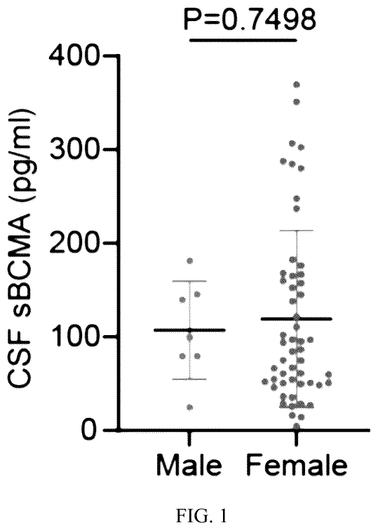

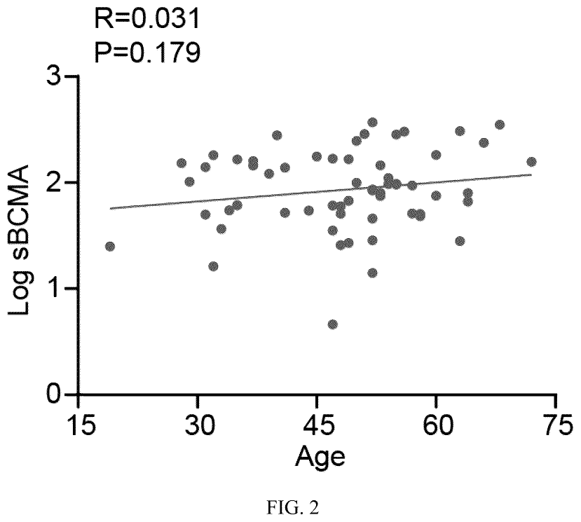

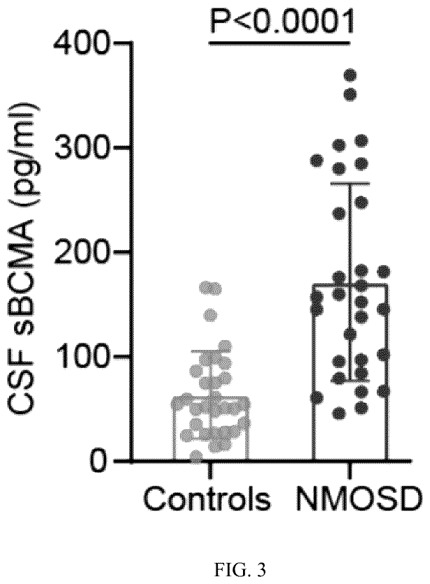

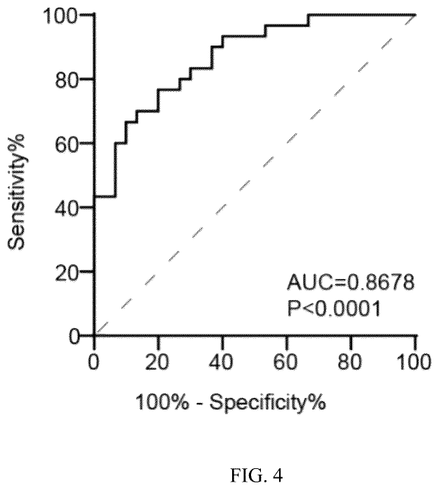

Specifically, the levels of sBCMA in cerebrospinal fluid of 30 NMOSD patients and in cerebrospinal fluid of control subjects matched for gender and age were tested in this disclosure. The test results showed that there was no statistical difference in the level of sBCMA in cerebrospinal fluid between male subjects and female subjects (P=0.7498) (FIG. 1), and there was no correlation between the age of the subjects and the level of sBCMA in cerebrospinal fluid (R=0.031, P=0.179) (FIG. 2). Furthermore, the levels of sBCMA in cerebrospinal fluid of NMOSD patients and control subjects were analyzed. The results showed that the levels of sBCMA in cerebrospinal fluid of NMOSD patients were significantly higher than those of control subjects (P<0.0001) (FIG. 3). In addition, as shown in FIG. 4, the ROC curve results showed that the area under the curve was AUC=0.8678, and there was a significant difference (P<0.0001). This indicated that sBCMA in cerebrospinal fluid could be used as a biomarker to assist the diagnosis of NMOSD, and it had good sensitivity and specificity.

### Example 2 sBCMA in Cerebrospinal Fluid as a Biomarker of Nerve Injury in NMOSD

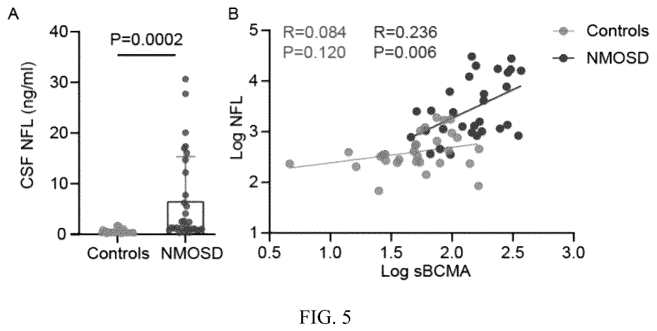

Neurofilament light chain (NFL), as a biomarker of nerve injury, is widely regarded as a dynamic reflection of irreversible nerve injury and the progress of diseases, and can be used as a biological index for the progress and monitoring of NMOSD diseases. In this example, based on the automated SIMOAHD-X platform, the level of NFL in the cerebrospinal fluid of the subjects for clinical study in Example 1 was quantified using hypersensitive SIMOA technology. The detection results showed that the level of NFL in cerebrospinal fluid of NMOSD patients was significantly higher than that of control subjects (P=0.0002). Further, this example analyzed the correlation between sBCMA and NFL in cerebrospinal fluid. As shown in FIG. 5, there was a positive correlation between the level of sBCMA and NFL in cerebrospinal fluid of NMOSD patients (R=0.236, P=0.006), but there was no correlation between the level of sBCMA and NFL in cerebrospinal fluid of control subjects (R=0.084, P=0.120). This suggested that the increase of the level of sBCMA in cerebrospinal fluid represented the aggravation of nerve injury, and the level of sBCMA in cerebrospinal fluid could indicate the severity of nerve injury. Therefore, the sBCMA in cerebrospinal fluid could be used as a biomarker for predicting nerve injury to reflect the degree of nerve injury in antibody-mediated CNS autoimmune diseases such as NMOSD.

### Example 3 sBCMA in Cerebrospinal Fluid as a Biomarker of Blood-Brain Barrier Injury in NMOSD

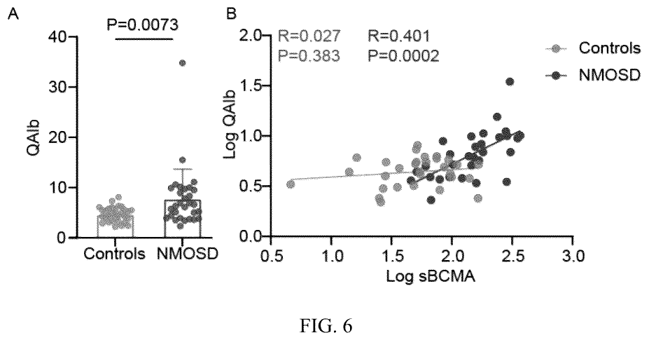

In NMOSD, interleukin-6, a proinflammatory cytokine, could destroy the integrity of blood-brain barrier (BBB), causing the increase of the penetration of AQP-4 antibodies and further causing subsequent neurotoxic reactions. The clinical research showed that the degree of BBB destruction was a reliable clinical marker for diagnosing NMOSD and its severity. The concentration gradient of protein between plasma and cerebrospinal fluid is caused by the function of blood-cerebrospinal fluid barrier. The ratio of the level of albumin (Alb) in cerebrospinal fluid to the level of albumin in serum is called an albumin quotient (QAlb), which could reflect the degree of damage to blood-cerebrospinal fluid barrier. The formula of QAlb was (Alb in cerebrospinal fluid/Alb in blood)×1000. The results were shown in FIG. 6, the level of QAlb of the NMOSD patients in the subjects for clinical study in Example 1 was significantly higher than that of the control subjects (P=0.0073). In this example, the correlation between the level of sBCMA in cerebrospinal fluid and QAlb was analyzed. The results showed that there was a positive correlation between the level of sBCMA in cerebrospinal fluid and QAlb in NMOSD patients (R=0.401, P=0.0002), but there was no correlation in the control subjects (R=0.027, P=0.383). This suggested that the increase of the level of sBCMA in cerebrospinal fluid represented the aggravation of blood-brain barrier breakdown, and the level of sBCMA in cerebrospinal fluid could be used as a biomarker to indicate the severity of breakdown to the blood-cerebrospinal fluid barrier function. Therefore, the level of sBCMA in cerebrospinal fluid could indicate the severity of breakdown to the blood-cerebrospinal fluid barrier in the pathogenesis of antibody-mediated CNS autoimmune diseases such as NMOSD.

### Example 4 sBCMA in Cerebrospinal Fluid as a Biomarker of Neuroinflammation in NMOSD

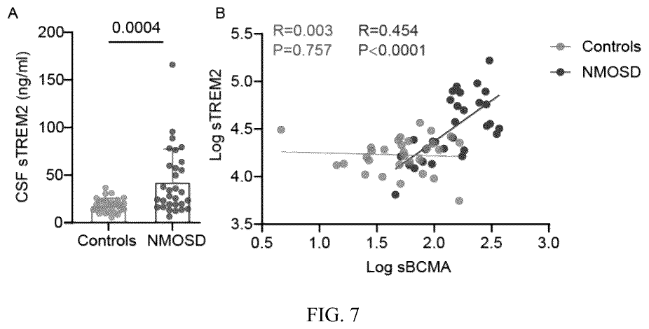

The increase of the level of soluble triggering receptors expressed on myeloid cells 2 (sTREM2) expressed in cerebrospinal fluid was related to the risk of NMOSD, and had a positive correlation with neuroinflammatory response. A human TREM2 ELISA kit (Abcam, ab224881) was adopted to detect the expression level of sTREM2 in cerebrospinal fluid of the subjects for clinical study in Example 1. As shown in FIG. 7, compared with the control subjects, the level of sTREM2 in NMOSD patients was significantly increased (P=0.0004). Similarly, the correlation between the level of sBCMA and the level of sTREM2 was analyzed in this example. The results showed that there was a positive correlation between the level of sBCMA in cerebrospinal fluid and sTREM2 in NMOSD patients (R=0.454, P<0.0001). However, this correlation did not exist in the control subjects (R=0.003, P=0.757). This indicated that the level of sBCMA in cerebrospinal fluid could feed back the degree of neuroinflammation, and it could be used as a biomarker to predict the level of neuroinflammation, which had a guiding role for early warning and auxiliary diagnosis of antibody-mediated central nervous system autoimmune diseases such as NMOSD.

### Example 5 Verification of the Application of sBCMA in Cerebrospinal Fluid as a Marker in the Diagnosis of NMOSD

In order to further confirm the reliability of sBCMA in cerebrospinal fluid as a biomarker of NMOSD, the following example included subjects for clinical study of 15 aquaporin-4 antibody-positive (AQP4-IgG positive) NMOSD patients and 15 serum AQP4 antibody-negative control subjects matched for gender and age as a validation cohort. Specific details are shown in the following table:

| | Control group (n = 15) | NMOSD (n = 15) |
|---|---|---|
| Female, n (%) | 12(80.0) | 12(80.0) |
| Age (years), median | 49 (27.0-69.0) | 52.0 (31.0-67.0) |
| (maximum/minimum) | | |

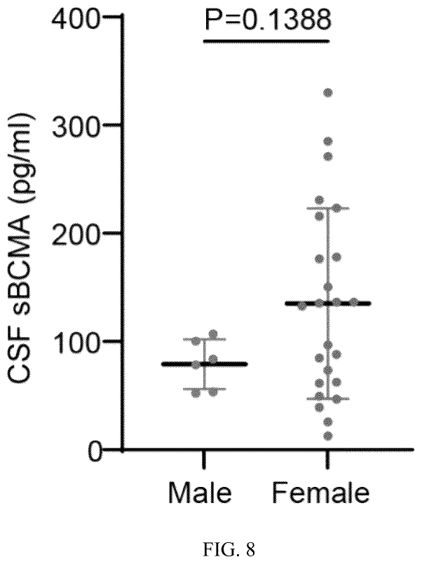

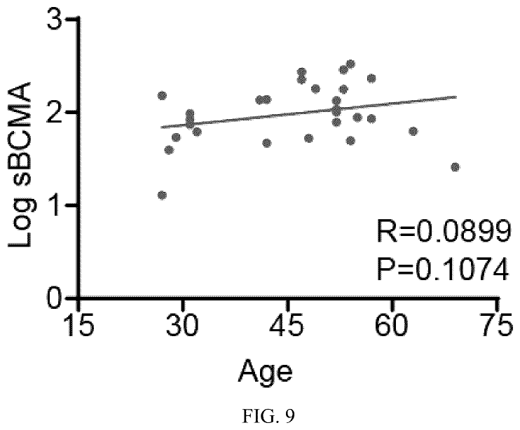

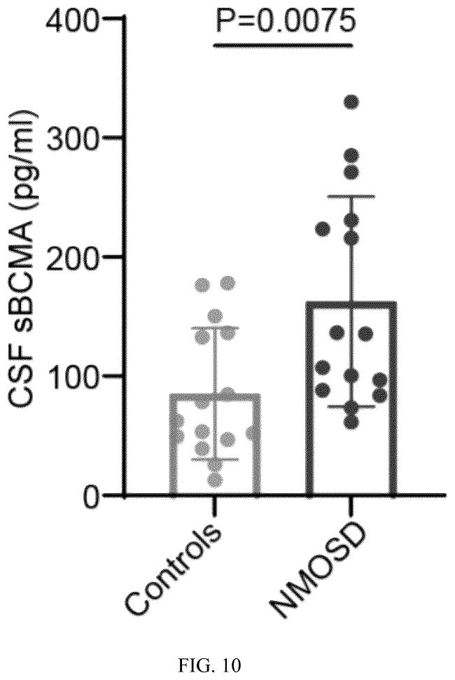

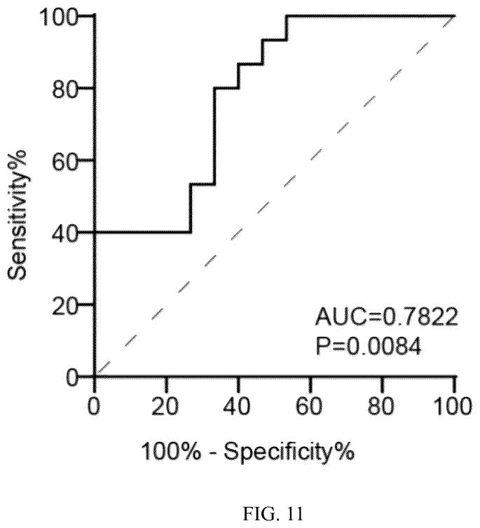

Specifically, the levels of sBCMA in cerebrospinal fluid of 15 NMOSD patients and 15 control subjects matched for gender and age in a validation cohort were further tested in this disclosure. The test results showed that there was no statistical difference in the level of sBCMA in cerebrospinal fluid between male subjects and female subjects (P=0.1388) (FIG. 8), and there was no correlation between the age of the subjects and the level of sBCMA in cerebrospinal fluid (R=0.0899, P=0.1074) (FIG. 9). Furthermore, the levels of sBCMA in cerebrospinal fluid of NMOSD patients and control subjects were analyzed. The results showed that the levels of sBCMA in cerebrospinal fluid of NMOSD patients were significantly higher than those of control subjects (P=0.0075) (FIG. 10). In addition, the ROC curve results showed that the area under the curve was AUC=0.7822, and there was a significant difference (P=0.0084) (FIG. 11). It was further confirmed that sBCMA in cerebrospinal fluid could be used as a biomarker of NMOSD to assist the diagnosis of NMOSD, and it had good sensitivity and specificity.

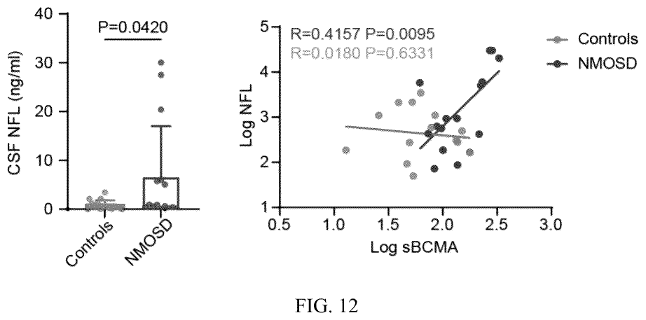

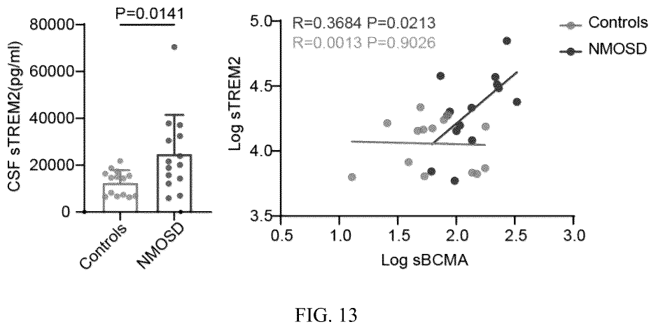

In addition, the levels of the nerve injury marker NFL and the neuroinflammation marker STREM2 in cerebrospinal fluid of the subjects in the validation cohort were detected in this disclosure. The detection results showed that the levels of NFL (P=0.0420) and sTREM2 (P=0.0141) in cerebrospinal fluid of NMOSD patients was significantly higher than those of control subjects. Further, the correlation between the sBCMA, NFL, and sTREM2 in cerebrospinal fluid was analyzed in this example. As shown in FIG. 12, there was a positive correlation between the level of sBCMA and NFL in cerebrospinal fluid of NMOSD patients (R=0.4157, P=0.0095), but there was no correlation between the level of sBCMA and NFL in cerebrospinal fluid of control subjects (R=0.0180, P=0.6331). This indicated that the level of sBCMA in cerebrospinal fluid could be used as a biomarker of the severity of nerve injury. As shown in FIG. 13, there was a positive correlation between the level of sBCMA in cerebrospinal fluid and the sTREM2 in NMOSD patients (R=0.3684, P=0.0213). However, this correlation did not exist in the control subjects (R=0.0013, P=0.9026). This indicated that the level of sBCMA in cerebrospinal fluid could feed back the degree of neuroinflammation, and it could be used as a biomarker of the level of neuroinflammation.

Based on the above analysis, sBCMA in cerebrospinal fluid can reflect the severity of nerve injury, blood-cerebrospinal fluid barrier breakdown, and inflammatory reaction of the central nervous system in NMOSD patients. Furthermore, based on the common pathogenesis characteristics of the antibody-mediated central nervous system autoimmune diseases, the level of sBCMA in cerebrospinal fluid can not only provide a powerful auxiliary means for the early diagnosis of NMOSD, but also provide excellent reference value for the clinical diagnosis and disease monitoring of other antibody-mediated central nervous system autoimmune diseases (such as myelin oligodendrocyte glycoprotein-IgG associated disorders, anti-N-methyl-D-aspartate receptor encephalitis, anti-leucine-rich glioma-inactivated protein 1 encephalitis, and anti-contactin associated protein-like 2 antibody autoimmune encephalitis).

What is claimed is:

1. An application of a reagent for detecting a biomarker sBCMA in preparation of a kit, wherein the kit is used for diagnosis of antibody-mediated central nervous system autoimmune diseases.

2. An application according to claim 1, wherein the kit is used for evaluating the severity of nerve injuries of patients with antibody-mediated central nervous system autoimmune diseases.

3. An application according to claim 1, wherein the kit is used for evaluating the degree of damage to blood-cerebrospinal fluid barrier of patients with antibody-mediated central nervous system autoimmune diseases.

4. An application according to claim 1, wherein the kit is used for evaluating the level of neuroinflammation of patients with antibody-mediated central nervous system autoimmune diseases.

5. The application according to claim 1, wherein the biomarker sBCMA is derived from cerebrospinal fluid.

6. The application according to claim 1, comprising the following steps: 1) determining the level of sBCMA in cerebrospinal fluid of subjects; and 2) carrying out significant differential analysis by comparing the level of sBCMA in the cerebrospinal fluid of the subjects with the level of sBCMA of control subjects matched for age and gender.

7. The application according to claim 1, wherein the antibody-mediated central nervous system autoimmune diseases are neuromyelitis optica spectrum disorders.
